(19) United States
(12) Patent Application Publication
(10) Pub. No.: US 2024/0428065 A1
(43) Pub. Date: Dec. 26, 2024

(54) LATENT REPRESENTATION LEARNING BASED ON MOLECULAR GRAPHS AND PRODUCTION RULES

(71) Applicant: International Business Machines Corporation, Armonk, NY (US)

(72) Inventors: AKIHIRO KISHIMOTO, Tokyo (JP); Hiroshi Kajino, Tokyo (JP)

(21) Appl. No.: 18/213,202

(22) Filed: Jun. 22, 2023

(51) Int. Cl. G06N 3/08; G06N 3/0455; G16C 20/50; G16C 20/70

(52) U.S. Cl. CPC G06N 3/08; G06N 3/0455; G16C 20/50; G16C 20/70

(57) ABSTRACT

One embodiment of the invention provides a computer-implemented method for training an autoencoder to learn one or more chemical properties. The method comprises providing, as input, to an encoder of the autoencoder, a molecular graph representing a molecular structure. The method further comprises receiving, as output, from a decoder of the autoencoder, a production rule sequence for producing a molecule description of the molecular structure. The method further comprises optimizing the autoencoder using a loss function and the production rule sequence.

### BACKGROUND

The field of embodiments of the invention generally relate to autoencoders.

An autoencoder is a type of artificial neural network used to learn efficient codings of unlabeled data (i.e., unsupervised learning). An autoencoder learns two functions: (i) an encoding function that transforms input data, and (ii) a decoding function that recreates the input data from encoded representation.

### SUMMARY

Embodiments of the invention generally relate to autoencoders, and more specifically, training an autoencoder to learn latent representations based on molecular graphs and production rules.

One embodiment of the invention provides a computer-implemented method for training an autoencoder to learn one or more chemical properties. The method comprises providing, as input, to an encoder of the autoencoder, a molecular graph representing a molecular structure. The method further comprises receiving, as output, from a decoder of the autoencoder, a production rule sequence for producing a molecule description of the molecular structure. The method further comprises optimizing the autoencoder using a loss function and the production rule sequence. Other embodiments include a system for training an autoencoder, and a computer program product for training an autoencoder.

### BRIEF DESCRIPTION OF THE DRAWINGS

The subject matter which is regarded as embodiments of the invention are particularly pointed out and distinctly claimed in the claims at the conclusion of the specification. The foregoing and other objects, features, and advantages of embodiments of the invention are apparent from the following detailed description taken in conjunction with the accompanying drawings in which:

FIG. 1 depicts a computing environment according to an embodiment of the present invention;

FIG. 2 illustrates an example computing architecture for implementing latent representation learning based on molecular graphs and production rules, in accordance with an embodiment of the invention;

FIG. 3 illustrates an example latent representation learning system in detail, in accordance with an embodiment of the invention;

FIG. 4 illustrates the latent representation learning system during training, in accordance with an embodiment of the invention;

FIG. 5 illustrates example molecular graphs, example production rule sequences, and example production rules, in accordance with an embodiment of the invention;

FIG. 6 is a flowchart for an example process for training an autoencoder to learn one or more chemical properties, in accordance with an embodiment of the invention; and

FIG. 7 is a flowchart for an example process for further training an autoencoder to learn one or more chemical properties, in accordance with an embodiment of the invention.

The detailed description explains the preferred embodiments of the invention, together with advantages and features, by way of example with reference to the drawings.

### DETAILED DESCRIPTION

Embodiments of the invention generally relate to autoencoders, and more specifically, training an autoencoder to learn latent representations based on molecular graphs and production rules. One embodiment of the invention provides a computer-implemented method for training an autoencoder to learn one or more chemical properties. The method comprises providing, as input, to an encoder of the autoencoder, a molecular graph representing a molecular structure. The method further comprises receiving, as output, from a decoder of the autoencoder, a production rule sequence for producing a molecule description of the molecular structure. The method further comprises optimizing the autoencoder using a loss function and the production rule sequence.

In at least some embodiments, a training dataset comprising a first molecule description in line notation of a first molecular structure is received as input, and the first molecule description is converted into a first molecular graph representing the first molecular structure. In at least some embodiments, a first production rule sequence of a context-free grammar (CFG) for the line notation is generated based on the first molecular graph. In at least some embodiments, the first molecular graph is encoded, via the encoder, into a latent representation, and the latent representation is decoded, via the decoder, into a second production rule sequence of the CFG for the line notation. In at least some embodiments, a comparison of the first production rule sequence and the second production rule sequence is performed in accordance with the loss function, and the encoder and the decoder are trained based on the comparison. These features contribute to the advantages of a trained autoencoder configured to encode a molecular graph representing a molecular structure into a latent representation of the molecular graph, and further configured to decode a latent representation of a molecular graph representing a molecular structure into a production rule sequence for producing a grammatically valid description of a valid molecular structure.

In at least some embodiments, the encoder comprises a first deep learning model, and the decoder comprises a second deep learning model. In at least some embodiments, the first deep learning model comprises a graph neural network (GNN), and the second deep learning model comprises a recurrent neural network (RNN). Unlike conventional autoencoders, the GNN learns latent representations of molecular graphs based on molecular graphs the GNN receives as input. By utilizing the GNN, the autoencoder can encode molecular graphs of molecular structures into latent representations of the molecular graphs. Unlike conventional autoencoders, the RNN learns production rule sequences based on latent representations of molecular graphs the RNN receives as input. By utilizing the RNN, the autoencoder can decode latent representations of molecular graphs into production rule sequences.

In at least some embodiments, a second molecular graph representing a second molecular structure is generated, and the second molecular graph is converted into a second molecule description in the line notation of the second molecular structure. In at least some embodiments, the first deep learning model and the second deep learning model are trained to minimize a difference quantified by the loss function, such that the second molecule description is grammatically valid and describes a valid molecular structure. In at least some embodiments, the loss function is a beta-variational autoencoder (beta-VAE) model.

In at least some embodiments, the first production rule sequence is further based on molecular hypergraph grammar. This ensures that any molecule description produced using the first production rule sequence is grammatically valid and describes a valid molecular structure. The autoencoder can also encode graphs representing proprietary or new molecular structures.

In at least some embodiments, the line notation is simplified molecular-input line-entry system (SMILES). Unlike conventional autoencoders, the autoencoder generates SMILES strings as output that are grammatically valid and describe a valid molecular structure.

Another embodiment of the invention provides a system for training an autoencoder to learn one or more chemical properties. The system comprises at least one processor and a non-transitory processor-readable memory device storing instructions that when executed by the at least one processor causes the at least one processor to perform operations. The operations include providing, as input, to an encoder of the autoencoder, a molecular graph representing a molecular structure. The operations further include receiving, as output, from a decoder of the autoencoder, a production rule sequence for producing a molecule description of the molecular structure. The operations further include optimizing the autoencoder using a loss function and the production rule sequence.

One embodiment of the invention provides a computer program product for training an autoencoder to learn one or more chemical properties, the computer program product comprising a computer readable storage medium having program instructions embodied therewith. The program instructions are executable by a processor to cause the processor to provide, as input, to an encoder of the autoencoder, a molecular graph representing a molecular structure. The program instructions are executable by the processor to further cause the processor to receive, as output, from a decoder of the autoencoder, a production rule sequence for producing a molecule description of the molecular structure. The program instructions are executable by the processor to further cause the processor to optimize the autoencoder using a loss function and the production rule sequence.

A molecular structure is the shape or configuration of a molecule, including the location of atoms, groups, or ions relative to one another in the molecule, as well as the number and location of chemical bonds. A molecule description comprises a description in line notation of a molecular structure of a molecule. A line notation represents a chemical structure (e.g., molecular structure) as a linear string of characters. A molecular graph comprises a graph structure representing a molecular structure of a molecule. A molecular graph is multi-dimensional (e.g., two-dimensional, three-dimensional, etc.).

SMILES is a specification in the form of a line notation for describing a chemical structure (e.g., molecular structure) using short ASCII strings (“SMILES strings”).

CFG is a formal grammar which is used to generate all possible strings in a given formal language and whose production rules can be applied to be nonterminal symbols regardless of context. A production rule sequence comprises a sequence of production rules of a CFG for a line notation (e.g., SMILES). A sequence of production rules can be represented as a sequence of integers (i.e., each integer represents a particular production rule).

Some conventional autoencoders for learning latent representations receive SMILES strings as input and attempt to generate identical SMILES strings as output. These conventional autoencoders, however, frequently decode SMILES strings that are grammatically invalid and cannot satisfy necessary structural constraints of molecules.

Other conventional autoencoders for learning latent representations may generate valid SMILES strings as output but such strings may not describe a valid molecular structure. These conventional autoencoders only understand molecular structures as text strings or production rule sequences, and do not utilize molecular graphs of molecular structures, resulting in the disadvantage of inaccurate understanding of molecular structures. Further, these conventional autoencoders may not be able to encode proprietary or new molecular structures if such structures are not available in training examples used to train the autoencoders.

Some conventional autoencoders for learning latent representations leverage junction trees which lose information on molecular structures and require inefficient, complicated neural networks for encoding/decoding.

One or more embodiments of the invention provide a framework for latent representation learning based on molecular graphs and production rules. In at least some embodiments, the framework comprises an autoencoder that includes a GNN for encoding and a RNN for decoding. The GNN learns latent representations of molecular graphs based on molecular graphs the GNN receives as input. The RNN learns production rule sequences based on latent representations of molecular graphs the RNN receives as input. The autoencoder generates production rule sequences corresponding to molecular graphs, and vice versa. The autoencoder generates SMILES strings as output that are grammatically valid and describe a valid molecular structure. The autoencoder can also encode molecular graphs of proprietary or new molecular structures.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

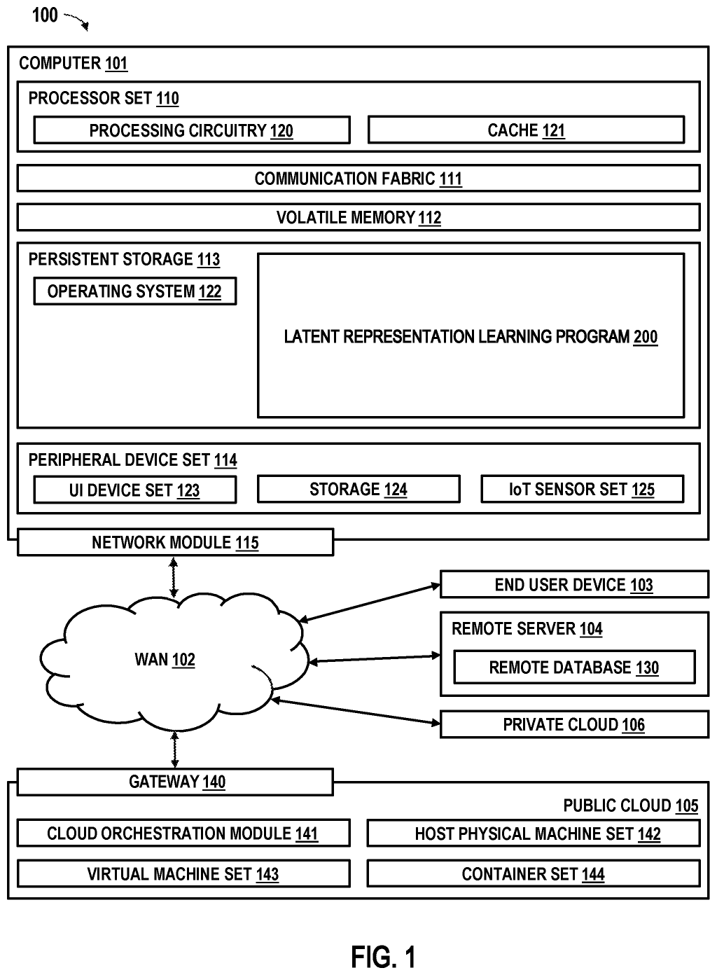

FIG. 1 depicts a computing environment 100 according to an embodiment of the present invention. Computing environment 100 contains an example of an environment for the execution of at least some of the computer code involved in performing the inventive methods, such as latent representation learning program 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 012 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

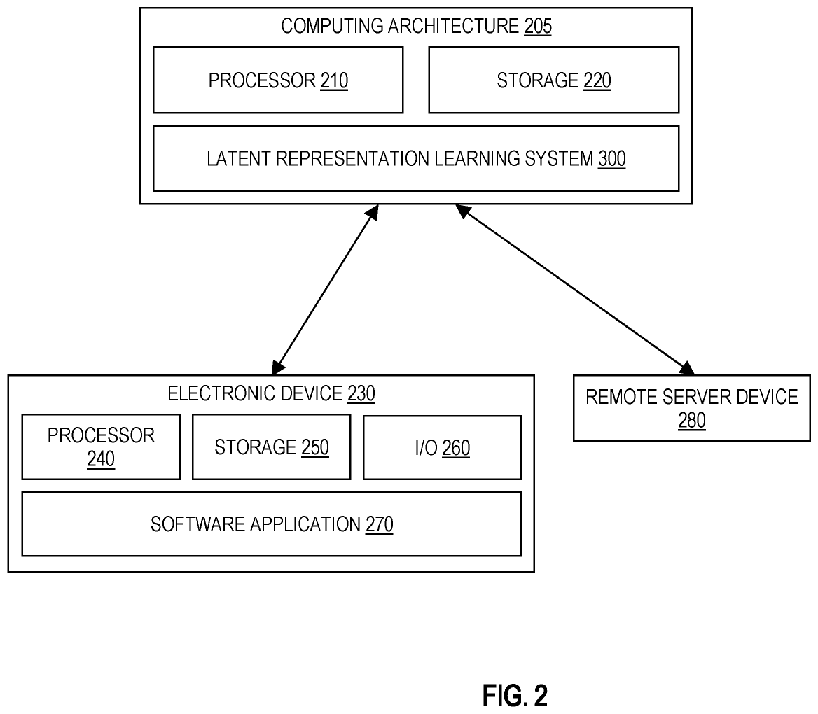

FIG. 2 illustrates an example computing architecture 205 for implementing latent representation learning based on molecular graphs and production rules, in accordance with an embodiment of the invention. In at least some embodiments, the computing architecture 205 is a centralized computing architecture. In another embodiment, the computing architecture 205 is a distributed computing architecture.

In at least some embodiments, the computing architecture 205 comprises computation resources such as, but not limited to, one or more processor units 210 and one or more storage units 220. One or more applications may execute/operate on the computing architecture 205 utilizing the computation resources of the computing architecture 205. In at least some embodiments, the applications on the computing architecture 205 include, but are not limited to, a latent representation learning system 300 for training an autoencoder. As described in detail later herein, the system 300 trains the autoencoder to learn effective latent representations (i.e., latent embeddings or latent space) based on molecular graphs and production rules. The resulting trained autoencoder may be utilized in several applications, such as chemical property prediction and molecular structure generation/optimization in the fields of chemical and/or material sciences.

In at least some embodiments, the system 300 is configured to exchange data with one or more electronic devices 230 and/or one or more remote server devices 280 over a connection (e.g., a wireless connection such as a Wi-Fi connection or a cellular data connection, a wired connection, or a combination of the two).

In at least some embodiments, an electronic device 230 comprises one or more computation resources such as, but not limited to, one or more processor units 240 and one or more storage units 250. One or more applications may execute/operate on an electronic device 230 utilizing the one or more computation resources of the electronic device 230 such as, but not limited to, one or more software applications 270 loaded onto or downloaded to the electronic device 230.

Examples of an electronic device 230 include, but are not limited to, a desktop computer, a mobile electronic device (e.g., a tablet, a smart phone, a laptop, etc.), a wearable device (e.g., a smart watch, etc.), an Internet of Things (IoT) device, etc.

In at least some embodiments, an electronic device 230 comprises one or more input/output (I/O) units 260 integrated in or coupled to the electronic device 230, such as a keyboard, a keypad, a touch interface, a display screen, etc. A user may utilize an I/O module 260 of an electronic device 230 to configure one or more user preferences, configure one or more parameters, provide user input, etc.

In at least some embodiments, the system 300 can be accessed or utilized by one or more online services hosted on a remote server device 280 and/or one or more software applications 270 operating on an electronic device 230.

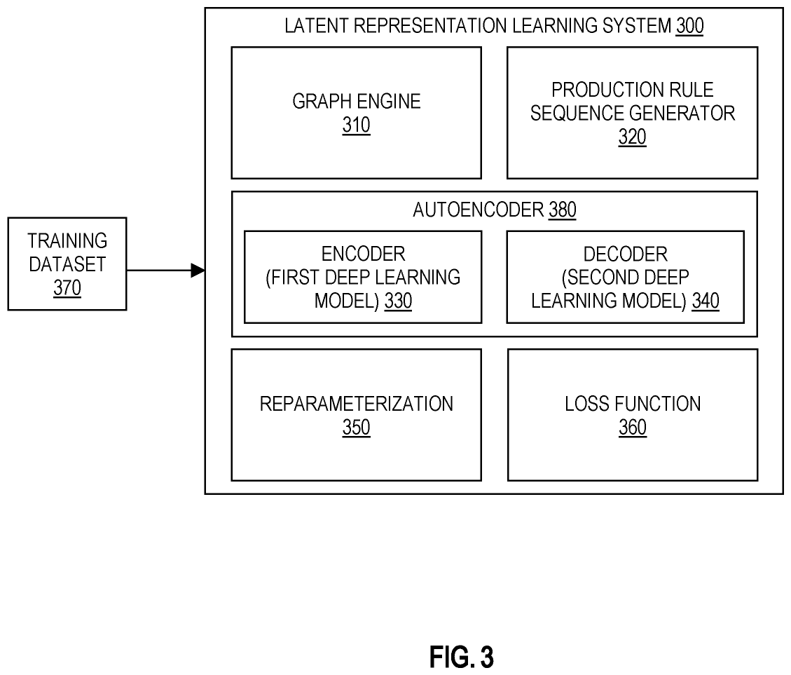

FIG. 3 illustrates an example latent representation learning system 300 for training an autoencoder 380 in detail, in accordance with an embodiment of the invention. In at least some embodiments, the system 300 comprises a graph engine 310 configured to perform operations including: (1) receiving, as input, a training dataset 370 comprising one or more molecule descriptions of one or more molecular structures, and (2) for each molecule description of each molecular structure received, converting the molecule description into a molecular graph of the molecular structure.

In at least some embodiments, each molecule description received by the graph engine 310 is in SMILES format (i.e., the graph engine 310 is a SMILES-based graph engine).

In at least some embodiments, the graph engine 310 is further configured to perform reverse or opposite operations including: (1) receiving, as input, a molecular graph of a molecular structure, and (2) converting the molecular graph into a molecule description of the molecular structure. In at least some embodiments, each molecule description converted by the graph engine 310 is in SMILES format.

In at least some embodiments, the system 300 comprises a production rule sequence generator 320 configured to perform operations including: (1) receiving, as input, a molecular graph of a molecular structure (e.g., from the graph engine 310), and (2) based on the molecular graph, generating a production rule sequence for producing a molecule description of the molecular structure. In at least some embodiments, each production rule sequence comprises a sequence of production rules of a CFG for SMILES.

A hypergraph is a generalization of a graph in which an edge can join any number of vertices. A hyperedge is connection between two or more vertices of a hypergraph. A hyperedge replacement grammar (HRG) is a CFG for generating a hypergraph. Molecular hypergraph grammar (MHG) is an HRG that always generates molecular hypergraphs. A molecular hypergraph models an atom by a hyperedge and a bond by a node.

In at least some embodiments, the production rule sequence generator 320 generates a production rule sequence by leveraging MHG, thereby ensuring that a molecule description in SMILES format produced using the production rule sequence comprises SMILES strings that are grammatically valid and describe a valid molecular structure.

A junction tree is a tree where nodes and edges are labeled with sets of variables. A reversible junction tree (RJT) is a tree representation of a molecule, where the RJT is reversely convertible to the molecule without external information. In another embodiment, the production rule sequence generator 320 generates a reversible junction tree (RJT) based on a molecular graph of a molecular structure received, and converts the RJT into a production rule sequence for producing a molecule description of the molecular structure by performing depth-first search.

In at least some embodiments, the product rule sequence generator 320 is further configured to perform reverse or opposite operations including: (1) receiving, as input, a production rule sequence for producing a molecule description of a molecular structure (e.g., from a decoder 340), and (2) based on the production rule sequence, generating a molecular graph of the molecular structure.

In at least some embodiments, the system 300 includes the autoencoder 380. The autoencoder 380 comprises an encoder 330. The encoder 330 comprises a first deep learning model configured to: (1) receive, as input, a molecular graph of a molecular structure (e.g., from the graph engine 310), and (2) encode the molecular graph into a latent vector representation (i.e., latent embedding or latent space) of the molecular graph.

In at least some embodiments, the first deep learning model of the encoder 330 is a GNN (i.e., the encoder 330 is a GNN-based encoder). A GNN can be directly applied to a graph (e.g., a molecular graph) to perform inference on data described by the graph. Examples of GNNs include, but are not limited to, graph isomorphism networks. By utilizing a GNN, the encoder 330 can encode molecular graphs of molecular structures that otherwise cannot be encoded via conventional autoencoders, such as a MHG-variational autoencoder (MHG-VAE), because of the lack of such structures in training examples. This provides the encoder 330 with the advantage of being able to perform downstream tasks involving proprietary or new molecular structures.

In at least some embodiments, the autoencoder 380 comprises a decoder 340. The decoder 340 comprises a second deep learning model configured to: (1) receive, as input, a latent vector representation of a molecular graph (e.g., encoded by the encoder 330 or reparameterized by a reparameterization unit 350), and (2) decode the latent vector representation into a production rule sequence for producing a molecule description of a molecular structure. In at least some embodiments, each production rule sequence decoded by the decoder 340 produces a molecule description in SMILES format.

In at least some embodiments, the second deep learning model of the decoder 340 is a RNN (i.e., the decoder 340 is a RNN-based encoder). A RNN is a type of artificial neural network which uses sequential data or time series data. Examples of RNNs include, but are not limited to, a gated recurrent unit (GRU), a long short-term memory (LSTM), etc. The RNN of the decoder 340 is configured to receive different inputs including: (1) a latent vector representation of a molecular graph, and (2) a production rule sequence for producing a molecule description of a molecular structure. This is unlike some conventional decoders that receive a latent vector representation of a production rule sequence instead (e.g., a sequence of integers representing a sequence of production rules) which ignores a molecular structure of a molecule.

In at least some embodiments, the system 300 comprises a reparameterization unit 350 configured to: (1) receive, as input, a latent vector representation of a molecular graph (e.g., encoded by the encoder 330), and (2) reparameterize the latent vector representation using one or more outputs (e.g., mean and standard deviation) of the first deep learning model of the encoder 330. In some embodiments the reparameterization unit 350 expresses the input variable as a deterministic variable that is a parameterized vector-valued function and that uses an auxiliary variable input with an independent marginal probability.

In at least some embodiments, the system 300 comprises a loss function unit 360 configured to: (1) receive, as input, a first production rule sequence that is generated based on a molecular graph (e.g., from the production rule sequence generator 320), (2) receive, as input, a second production rule sequence that is decoded from a latent vector representation (e.g., from the decoder 340), (3) perform a comparison of the first production rule sequence and the second production rule sequence in accordance with a loss function, and (4) based on the comparison, train both the first deep learning model of the encoder 330 and the second deep learning model of the decoder 340. The first production rule sequence is an input to the encoder 330 and represents an expected/target outcome, whereas the second production rule sequence is an output of the decoder 340 and represents a predicted outcome. Therefore, the loss function quantifies a difference between the expected/target outcome and the predicted outcome. The first deep learning model (e.g., GNN) of the encoder 330 and the second deep learning model (e.g., RNN) of the decoder 340 are trained to minimize the difference.

In at least some embodiments, the GNN of the encoder 330 is trained to learn effective latent representations (i.e., latent embeddings or latent space) on molecular structures, and the RNN of the decoder 340 is trained to learn production rule sequences. In at least some embodiments, because of constraints on the applicability of each production rule sequence (i.e., grammatically valid and describe a valid molecular structure), the search space of the decoder 340 is reduced.

In at least some embodiments, the loss function is a beta-VAE model. A variational autoencoder provides a probabilistic manner for describing an observation in latent space. Beta-VAE is a type of variational autoencoder for automated discovery of disentangled latent representations (i.e., latent factors).

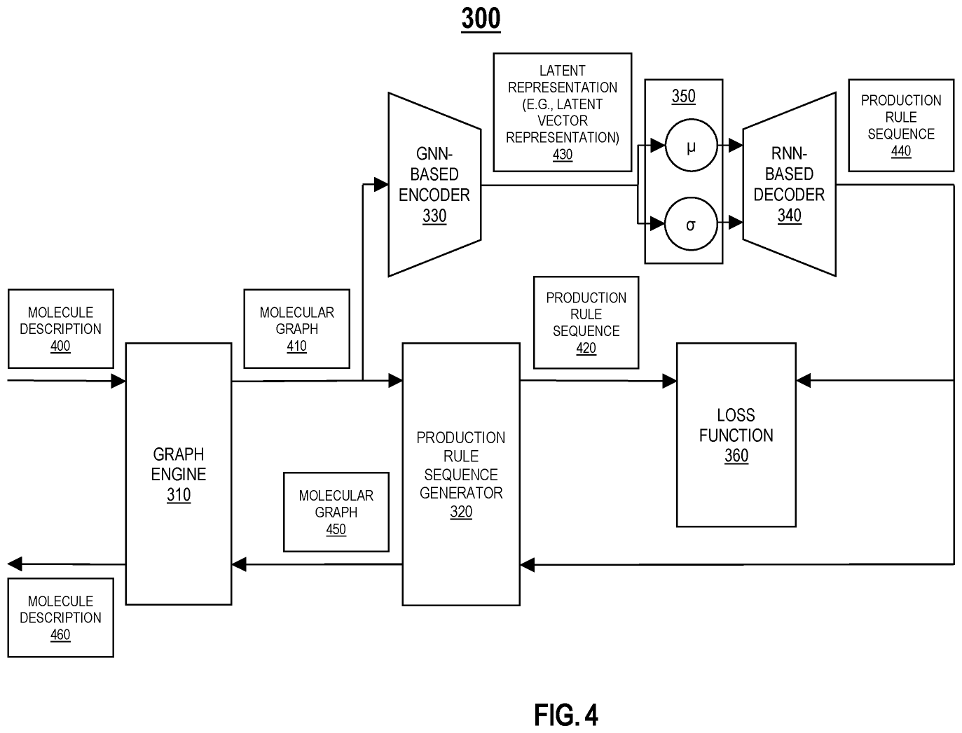

FIG. 4 illustrates the latent representation learning system 300 during training, in accordance with an embodiment of the invention. In at least some embodiments, the graph engine 310 is a SMILES-based graph engine. For example, RDKit, an open-source toolkit for cheminformatics, can be used as a SMILES-based graph engine. During the training, the graph engine 310 receives, as input, a training dataset 370 (FIG. 3) comprising one or more molecule descriptions 400 in SMILES format (i.e., comprising SMILES strings). For each molecule description 400 of the training dataset, the graph engine 310 converts the molecule description 400 into a corresponding molecular graph 410.

The production rule sequence generator 320 receives, as input, each molecular graph 410 converted by the graph engine 310. For each molecular graph 410 received, the production rule sequence generator 320 generates a corresponding production rule sequence 420 for producing a molecule description in SMILES format.

In at least some embodiments, the encoder 330 is a GNN-based encoder. The encoder 330 receives, as input, each molecular graph 410 converted by the graph engine 310. For each molecular graph 410 received, the encoder 330 encodes the molecular graph 410 into a corresponding latent vector representation 430 of the molecular graph.

The reparameterization unit 350 receives, as input, each latent vector representation 430 encoded by the encoder 330. For each latent vector representation 430 received, the reparameterization unit 350 reparameterizes the latent vector representation using one or more outputs (e.g., mean μ and standard deviation σ) of the GNN of the encoder 330.

In at least some embodiments, the decoder 340 is a RNN-based encoder. The decoder 340 receives, as input, each latent vector representation 430 reparameterized by the reparameterization unit 350. For each latent vector representation 430 received, the decoder 340 decodes the latent vector representation 430 into a corresponding production rule sequence 440.

The production rule sequence generator 320 receives, as input, each production rule sequence 440 decoded by the decoder 340. For each production rule sequence 440 received, the production rule sequence generator 320 generates a corresponding molecular graph 450.

The graph engine 310 receives, as input, each molecular graph 450 generated by the production rule sequence generator 320. For each molecular graph 450 received, the graph engine 310 converts the molecular graph 450 into a corresponding molecule description 460.

In at least some embodiments, the training operates iteratively, i.e., each iteration of the training involves training the system 300 using a particular molecule description 400 of the training dataset. During each iteration of the training, the loss function unit 360 performs a comparison of a production rule sequence 420 generated by the production rule sequence generator 320 (during the iteration) and a production rule sequence 440 decoded by the decoder 340 (during the iteration) in accordance with a loss function. The GNN of the encoder 330 and the RNN of the decoder 340 are then trained based on the comparison. The GNN of the encoder 330 and the RNN of the decoder 340 are trained to minimize a difference (quantified by the loss function) between the production rule sequences 420 and 440, such that each molecule description 460 converted by the graph engine 310 is grammatically valid (in SMILES format) and describes a valid molecular structure.

After the training, the resulting trained autoencoder 380 (FIG. 3) is configured to encode (via the trained GNN-based encoder 330) a molecular graph into a latent representation of the molecular graph, and further configured to decode (via the trained RNN-based decoder 340) a latent representation of a molecular graph into a production rule sequence for producing a grammatically valid description of a valid molecular structure.

In at least some embodiments, after the autoencoder 380 is trained, the encoder 330 can be used to address important tasks in chemical and/or material sciences. For example, given a training dataset comprising molecule descriptions of molecular structures of molecules and each of the molecules highest occupied molecular orbital-least unoccupied molecular orbital (HOMO-LUMO) energy, the encoder 330 can be used to generate a model for predicting HOMO-LUMO energy as follows: (1) prepare a support vector regression (SVR) as a model for chemical property prediction, (2) for each training example, create a corresponding pair (p1, p2), wherein p1 is the output of the encoder 330 of a molecule, and p2 is the HOMO-LUMO energy of that molecule, and (3) train the SVR using a collection of pairs (p1, p2).

As another example, given a solute and a solvent, the encoder 330 can be used in a downstream prediction task involving predicting Amax, wherein Amax is a maximum wavelength of absorption spectrum. This prediction task plays an important role in material discovery, such as developing new dyes. Before performing this prediction task, the autoencoder 380 is trained using a training dataset comprising molecule descriptions of molecular structures of about 980,000 molecules. The molecule descriptions are obtainable from an accessible public information database which provides a comprehensive database of chemical molecules. In one example, of 17,243 molecules prepared for this prediction task, the encoder 330 could successfully encode all of the molecules prepared. By comparison, the conventional autoencoder MHG-VAE could encode only 9,552 of the molecules prepared. Of the 9,552 molecules that MHG-VAE could encode only, 8563 molecules were used as a training set and 989 molecules were used as a validation set for the prediction task. Root Mean Square Error (RMSE) is the standard deviation of residuals (i.e., prediction errors). For the validation set, the autoencoder 380 has a RMSE of 22.73, whereas the conventional autoencoder MHG-VAE has a RSME of 32.53.

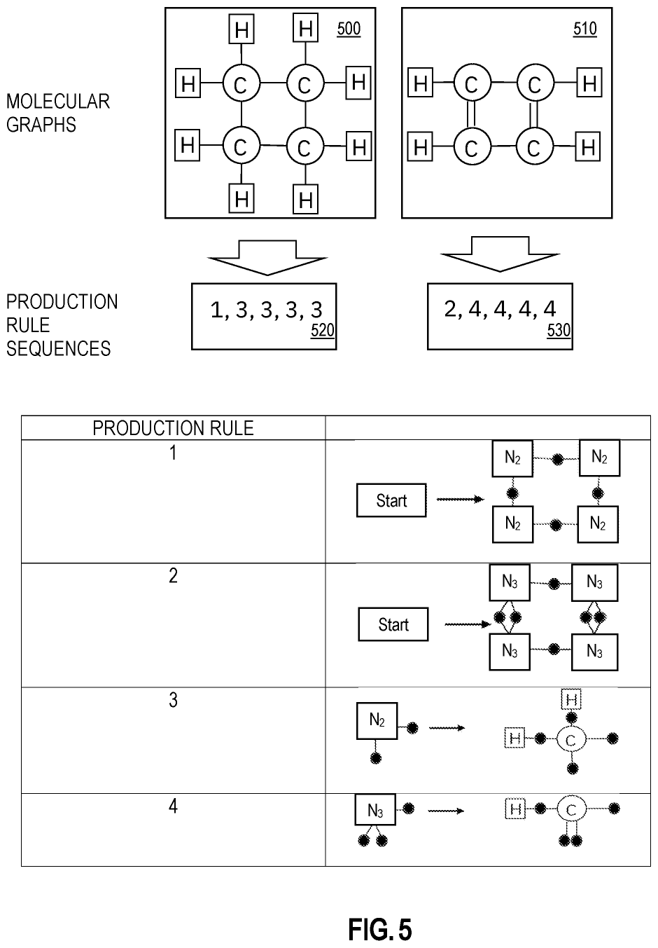

FIG. 5 illustrates example molecular graphs, example production rule sequences, and example production rules, in accordance with an embodiment of the invention. As shown in FIG. 5, a first production rule sequence 520 is generated (e.g., via the production rule sequence generator 320) based on a first molecular graph 500 representing a first molecular structure of a first molecule. As also shown in FIG. 5, a second production rule sequence 530 is generated (e.g., via the production rule sequence generator 320) based on a second molecular graph 510 representing a second molecular structure of a second molecule. Both the first molecule and the second molecule are hydrocarbons consisting entirely of carbon and hydrogen (e.g., the first molecule is alkane, and the second molecule is alkene).

As shown in FIG. 5, the first production rule sequence 520 comprises a first sequence of integers (1, 3, 3, 3, 3) representing a first production rule sequence for producing a first molecule description of the first molecule. As also shown in FIG. 5, the second production rule sequence 530 comprises a second sequence of integers (2, 4, 4, 4, 4) representing a second production rule sequence for producing a second molecule description of the second molecule. Specifically, integers ‘1’ and ‘3’ in the first sequence of integers represent a first production rule and a third production rule, respectively, and integers ‘2’ and ‘4’ in the second sequence of integers represent a second production rule and a fourth production rule, respectively.

As shown in FIG. 5, the first production rule defines a molecule as comprising four N₂ components arranged in a particular structure and in which all bonds are single. The third production rule defines a N₂ component as comprising two hydrogen atoms joined to a carbon atom and in which all bonds are single.

As shown in FIG. 5, the second production rule defines a molecule as comprising four N₃ components arranged in a particular structure and in which some bonds are single and some other bonds are double. The fourth production rule defines a N₃ component as comprising one hydrogen atom joined to a carbon atom and in which some bonds are single and some other bonds are double.

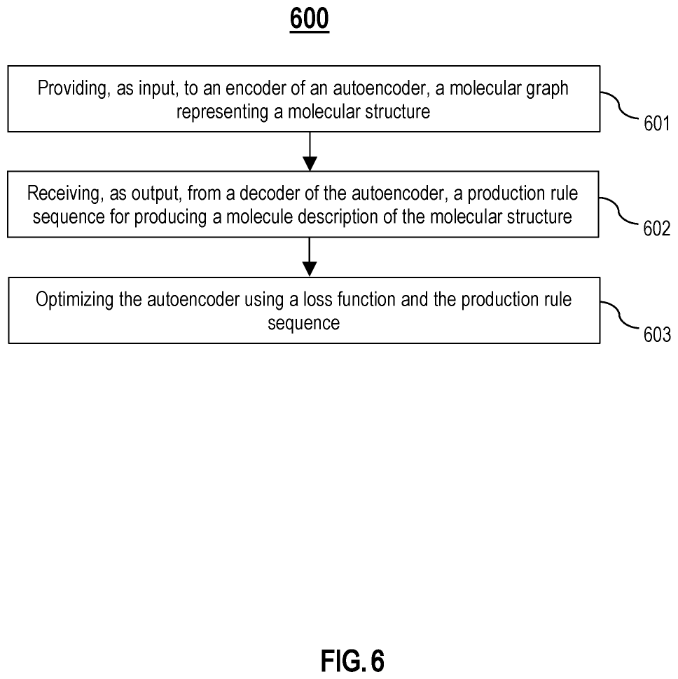

FIG. 6 is a flowchart for an example process 600 for training an autoencoder to learn one or more chemical properties, in accordance with an embodiment of the invention. Process block 601 includes providing, as input, to an encoder of an autoencoder, a molecular graph representing a molecular structure. Process block 602 includes receiving, as output, from a decoder of the autoencoder, a production rule sequence for producing a molecule description of the molecular structure. Process block 603 includes optimizing the autoencoder using a loss function (e.g., beta-VAE model) and the production rule sequence.

In at least some embodiments, process blocks 601-603 are performed by one or more components of the system 300.

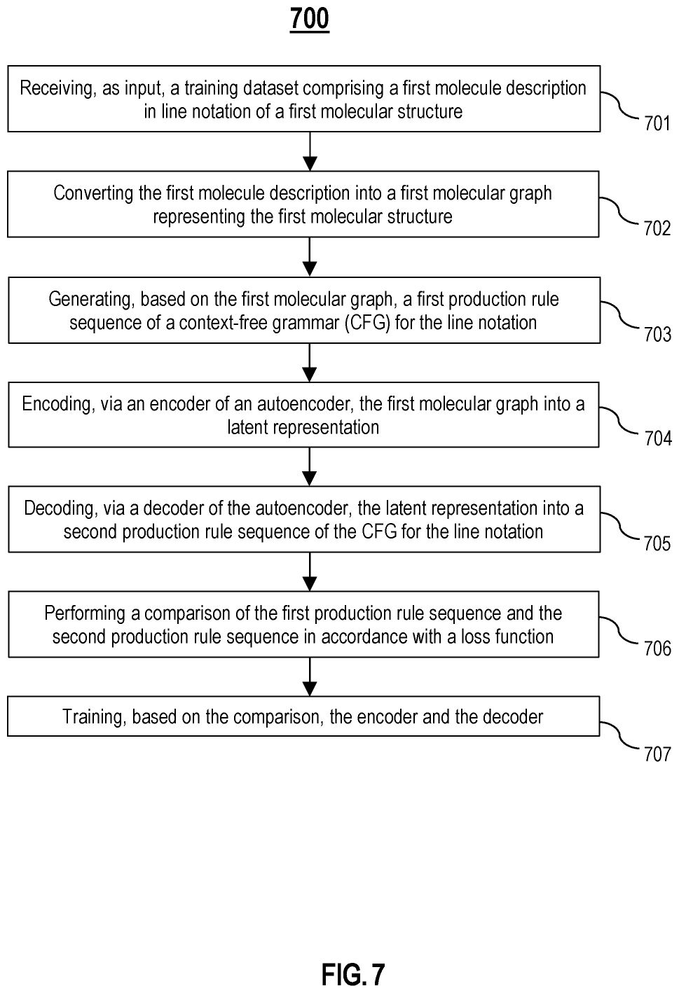

FIG. 7 is a flowchart for an example process 700 for further training an autoencoder to learn one or more chemical properties, in accordance with an embodiment of the invention. Process block 701 includes receiving, as input, a training dataset comprising a first molecule description (i.e., molecule description) in line notation (e.g., SMILES) of a first molecular structure (i.e., molecular structure). Process block 702 includes converting the first molecule description into a first molecular graph (i.e., molecular graph) representing the first molecular structure. Process block 703 includes generating, based on the first molecular graph, a first production rule sequence (i.e., production rule sequence) of a CFG for the line notation. Process block 704 includes encoding, via an encoder (e.g., GNN) of an autoencoder, the first molecular graph into a latent representation. Process block 705 includes decoding, via a decoder (e.g., RNN) of the autoencoder, the latent representation into a second production rule sequence of the CFG for the line notation. Process block 706 includes performing a comparison of the first production rule sequence and the second production rule sequence in accordance with a loss function (e.g., beta-VAE model). Process block 707 includes training, based on the comparison, the encoder and the decoder.

In at least some embodiments, process blocks 701-707 are performed by one or more components of the system 300.

From the above description, it can be seen that embodiments of the invention provide a system, computer program product, and method for implementing the embodiments of the invention. Embodiments of the invention further provide a non-transitory computer-useable storage medium for implementing the embodiments of the invention. The non-transitory computer-useable storage medium has a computer-readable program, wherein the program upon being processed on a computer causes the computer to implement the steps of embodiments of the invention described herein. References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. § 112(f), unless the element is expressly recited using the phrase “means for” or “step for.”

The terminology used herein is for the purpose of describing particular embodiments of the invention only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed.

The descriptions of the various embodiments of the invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for training an autoencoder to learn one or more chemical properties, comprising:
providing, as input, to an encoder of the autoencoder, a molecular graph representing a molecular structure;
receiving, as output, from a decoder of the autoencoder, a production rule sequence for producing a molecule description of the molecular structure; and
optimizing the autoencoder using a loss function and the production rule sequence.

2. The computer-implemented method of claim 1, further comprising:
receiving, as input, a training dataset comprising a first molecule description in line notation of a first molecular structure;
converting the first molecule description into the molecular graph, the molecular graph being a first molecular graph that represents the first molecular structure;
generating, based on the first molecular graph, a first production rule sequence of a context-free grammar (CFG) for the line notation;
encoding, via the encoder, the first molecular graph into a latent representation; and
decoding, via the decoder, the latent representation into a second production rule sequence of the CFG for the line notation, wherein the second production rule sequence is the production rule sequence received as output from the decoder;
wherein the optimization of the autoencoder comprises:
performing a comparison of the first production rule sequence and the second production rule sequence in accordance with the loss function; and
training, based on the comparison, the encoder and the decoder.

3. The computer-implemented method of claim 1, wherein the encoder comprises a first deep learning model, and the decoder comprises a second deep learning model.

4. The computer-implemented of claim 3, wherein the first deep learning model comprises a comprises a graph neural network (GNN), and the second deep learning model comprises a recurrent neural network (RNN).

5. The computer-implemented of claim 2, wherein the line notation is simplified molecular-input line-entry system (SMILES).

6. The computer-implemented method of claim 2, further comprising:
generating, based on the second production rule sequence, a second molecular graph representing a second molecular structure; and
converting the second molecular graph into a second molecule description in the line notation of the second molecular structure.

7. The computer-implemented method of claim 6, wherein the encoder and the decoder are trained to minimize a difference quantified by the loss function, such that the second molecule description is grammatically valid and describes a valid molecular structure.

8. The computer-implemented method of claim 1, wherein the loss function is a beta-variational autoencoder (beta-VAE) model.

9. The computer-implemented method of claim 2, wherein the first production rule sequence is further based on molecular hypergraph grammar.

10. A system for training an autoencoder to learn one or more chemical properties, comprising:
at least one processor; and
a non-transitory processor-readable memory device storing instructions that when executed by the at least one processor causes the at least one processor to perform operations including:
providing, as input, to an encoder of the autoencoder, a molecular graph representing a molecular structure;
receiving, as output, from a decoder of the autoencoder, a production rule sequence for producing a molecule description of the molecular structure; and
optimizing the autoencoder using a loss function and the production rule sequence.

11. The system of claim 10, wherein the operations further include:
receiving, as input, a training dataset comprising a first molecule description in line notation of a first molecular structure;
converting the first molecule description into the molecular graph, the molecular graph being a first molecular graph that represents the first molecular structure;
generating, based on the first molecular graph, a first production rule sequence of a context-free grammar (CFG) for the line notation;
encoding, via the encoder, the first molecular graph into a latent representation; and
decoding, via the decoder, the latent representation into a second production rule sequence of the CFG for the line notation, wherein the second production rule sequence is the production rule sequence received as output from the decoder;
wherein the optimization of the autoencoder comprises:
performing a comparison of the first production rule sequence and the second production rule sequence in accordance with the loss function; and
training, based on the comparison, the encoder and the decoder.

12. The system of claim 10, wherein the encoder comprises a first deep learning model, and the decoder comprises a second deep learning model.

13. The system of claim 12, wherein the first deep learning model comprises a graph neural network (GNN), and the second deep learning model comprises a recurrent neural network (RNN).

14. The system of claim 11, wherein the line notation is simplified molecular-input line-entry system (SMILES).

15. The system of claim 11, wherein the operations further include:
generating, based on the second production rule sequence, a second molecular graph representing a second molecular structure; and
converting the second molecular graph into a second molecule description in the line notation of the second molecular structure.

16. The system of claim 15, wherein the encoder and the decoder are trained to minimize a difference quantified by the loss function, such that the second molecule description is grammatically valid and describes a valid molecular structure.

17. The system of claim 10, wherein the loss function is a beta-variational autoencoder (beta-VAE) model.

18. The system of claim 11, wherein the first production rule sequence is further based on molecular hypergraph grammar.

19. A computer program product for training an autoencoder to learn one or more chemical properties, the computer program product comprising a computer readable storage medium having program instructions embodied therewith, the program instructions executable by a processor to cause the processor to:
provide, as input, to an encoder of the autoencoder, a molecular graph representing a molecular structure;
receive, as output, from a decoder of the autoencoder, a production rule sequence for producing a molecule description of the molecular structure; and
optimize the autoencoder using a loss function and the production rule sequence.

20. The computer program product of claim 19, wherein the program instructions executable by the processor further cause the processor to:
receive, as input, a training dataset comprising a first molecule description in line notation of a first molecular structure;
convert the first molecule description into the molecular graph, the molecular graph being a first molecular graph that represents the first molecular structure;
generate, based on the first molecular graph, a first production rule sequence of a context-free grammar (CFG) for the line notation;
encode, via the encoder, the first molecular graph into a latent representation; and
decode, via the decoder, the latent representation into a second production rule sequence of the CFG for the line notation, wherein the second production rule sequence is the production rule sequence received as output from the decoder;
wherein the optimization of the autoencoder by the processor further cause the processor to:
perform a comparison of the first production rule sequence and the second production rule sequence in accordance with the loss function; and
train, based on the comparison, the encoder and the decoder.
